(19) United States
(12) Patent Application Publication
(10) Pub. No.: US 2024/0189660 A1
(43) Pub. Date: Jun. 13, 2024

(54) FILTERING MASK

(71) Applicant: Yung-Jen CHEN, Kaohsiung City (TW)

(72) Inventor: Yung-Jen CHEN, Kaohsiung City (TW)

(21) Appl. No.: 18/502,016

(22) Filed: Nov. 5, 2023

(30) Foreign Application Priority Data
Dec. 13, 2022 (TW) 111147878

(51) Int. Cl. A63B 23/02

(52) U.S. Cl. CPC A63B 23/02

(57) ABSTRACT

A filtering mask includes a first filter pack provided with a filtration cartridge, two ventilation portions, a conduit, and a mask tube, wherein the filtration cartridge is disposed between the two ventilation portions, and the filtration cartridge is coupled in fluid communication with the conduit; a second filter pack provided with a filtration medium and a wrapper; wherein the filtration medium is disposed within the wrapper that is arranged around the conduit and the mask tube, and the second filter pack and the first filter pack are configured to be movably combined; and a supporting member detachably disposed between the first filter pack and the second filter pack.

### CROSS REFERENCE TO RELATED APPLICATIONS

This application claims the priority of Taiwan Patent Applications No. 111147878, titled as “FILTERING MASK”, filed on Dec. 13, 2022, the disclosures of which are incorporated herein by reference.

### FIELD OF THE INVENTION

The present disclosure relates to the technical field of air purifiers, and specifically to a filtering mask that is an air filtering device that can be provided in air purification devices and worn on human faces.

### BACKGROUND OF THE INVENTION

According to statistical results, the fatal reasons for building fires mainly include inhalation of poisonous gas resulting in suffocation and coma.

It is known that smoke-proof filtering masks and air purification devices are products with different purposes and cannot be universally used because the filtering methods and materials of the smoke-proof filtering masks and the air purification device are different.

It is common to have an air purification device in the family space. However, it is rare for the family space to be provided with a smoke-proof filtering mask required for the fire escape. The reason for the above is that the smoke-proof filtering mask needs to focus on a solid smoke-filtering function. In contrast, the air purification device usually focuses on dust removal and/or deodorization functions. Due to different functional orientations, it is difficult for the filter material with a smoke-proof function and the filter material required by the air purification device to be universally used. Although there have been some products with single or integrated functions in the past, they still need to be improved.

Given the above, it is necessary to provide a technical solution different from the past to solve the problems in the prior art.

### SUMMARY OF THE INVENTION

An object of the present disclosure is to provide a filtering mask applicable to ventilation at a fixed position with a single filter pack and being worn for filtering the air with dual filter assemblies.

To achieve the purpose mentioned above, one aspect of the present disclosure provides a filtering mask, which includes: a first filter pack provided with a filtration cartridge, two ventilation portions, a conduit, and a mask tube, wherein the filtration cartridge is disposed between the two ventilation portions, and the filtration cartridge is coupled in fluid communication with the conduit; a second filter pack provided with a filtration medium and a wrapper, wherein the filtration medium is disposed within the wrapper that is arranged around the conduit and the mask tube, and the second filter pack and the first filter pack are configured to be movably combined; and a supporting member detachably disposed between the first filter pack and the second filter pack.

In an embodiment of the present disclosure, in a first configuration mode, the supporting member is disposed between the first filter pack and the second filter pack, and the filtration medium is disposed within the wrapper in a fluid-closed manner; in a second configuration mode, the supporting member is away from the first filter pack and the second filter pack, and the conduit and the mask tube break the wrapper to make the filtration medium be coupled in fluid communication between the conduit and the mask tube.

In an embodiment of the present disclosure, the two ventilation portions include an air inlet portion and an air outlet portion, and in the first configuration mode, the filtration cartridge is coupled in fluid communication between the air inlet portion and the air outlet portion; in the second configuration mode, the filtration cartridge is coupled in fluid communication between the air inlet portion and the conduit.

In an embodiment of the present disclosure, the filtration medium is shaped as an arc-shaped filtration medium, in which a connection portion is provided between two ends of the arc-shaped filtration medium, wherein one end of the conduit is configured to be toward one of the two ends of the arc-shaped filtration medium, and one end of the mask tube is configured to be toward the connection portion of the arc-shaped filtration medium.

In an embodiment of the present disclosure, the first filter pack includes a housing, the filtration cartridge is disposed in the housing, the two ventilation portions are defined on the housing in different directions, and the housing is defined with a first circulation port that is coupled with the conduit in fluid communication; the wrapper includes a packaging assembly and two sealing membranes, the filtration medium is disposed within the packaging assembly, and the packaging assembly is defined with a second circulation port and a third circulation port, wherein the second circulation port and the conduit are aligned in an airflow path, the third circulation port and the mask tube are aligned in the airflow path, and the two sealing membranes are mounted on the second circulation port and the third circulation port.

In an embodiment of the present disclosure, the housing is provided with a plurality of first move-guiding elements, and the packaging assembly includes a move-guiding base, a seat, and a cover, wherein the move-guiding base has a plurality of second move-guiding elements that are slidably combined with the plurality of first move-guiding elements, the seat is mounted on the move-guiding base in a straddling manner and carries the filtration medium, and the cover covers the filtration medium and is coupled with the seat.

In an embodiment of the present disclosure, the move-guiding base has a substrate that is connected to the plurality of second move-guiding elements and defined with a through hole, wherein the through hole and the third circulation port are aligned in the airflow path, and during the plurality of first move-guiding elements and the plurality of second move-guiding elements linearly move relative to each other, one end of the mask tube passes through the through hole and the third circulation port.

In an embodiment of the present disclosure, the seat has a top and two shoulders, the second circulation port is defined on one of the two shoulders, and the third circulation port is defined on the top.

In an embodiment of the present disclosure, at least one elastic element is disposed between the housing and the substrate, and the at least one elastic member is arranged around the plurality of first move-guiding elements and the plurality of second move-guiding elements.

In an embodiment of the present disclosure, one of two ends of the conduit is configured to form a first tip toward the second circulation port; one of two ends of the mask tube is configured to form a second tip toward the third circulation port.

In an embodiment of the present disclosure, the other of the two ends of the mask tube is provided with a breathing aid.

In an embodiment of the present disclosure, the first filter pack includes a deformable body, the deformable body is disposed in the housing, the filtration cartridge is disposed within the deformable body, and the deformable body is configured to be deformable to change a rate of air flowing through the filtration cartridge.

In an embodiment of the present disclosure, the deformable body includes a frame and two flow-guiding sheets, and the filtration cartridge is disposed within the frame that has two openings adjacent to two sides of the filtration cartridge, wherein the two flow-guiding sheets are configured to be pivotally connected to the frame, and the two openings are in an open-up state or a closed state as the two flow-guiding sheets pivot relative to the frame.

In an embodiment of the present disclosure, the two flow-guiding sheets include a first flow-guiding sheet and a second flow-guiding sheet, wherein the first flow-guiding sheet is defined with a first air delivery portion, the second flow-guiding sheet is defined with a second air delivery portion, the first air delivery portion and the second air delivery portion are adjacent to the two ends of the filtration cartridge, and the second air delivery portion and the first circulation port are aligned in the airflow path.

In an embodiment of the present disclosure, the housing is defined with a slit, and the second flow-guiding sheet is provided with a baffle that is arranged around the second air delivery portion; in a first configuration mode, the first flow-guiding sheet and the second flow-guiding sheet cause the two openings to be in the open-up state, the baffle passes through the slit, and the filtration cartridge is coupled in fluid communication between the two ventilation portions; in a second configuration mode, the first flow-guiding sheet and the second flow-guiding sheet cause the two openings to be in the closed state, and the filtration cartridge is coupled in fluid communication between the first air delivery portion and the second air delivery portion.

In an embodiment of the present disclosure, the housing is defined with two strip-shaped coupling holes, and the two flow-guiding sheets are respectively provided with a strip-shaped guiding portion, wherein each of the two strip-shaped coupling holes is configured to allow a positioning pin to pass through slidably, wherein the positioning pin is slidably combined with the strip-shaped guiding portion, such that an intersection is formed between the strip-shaped guiding portion and one of the two strip-shaped coupling holes.

In an embodiment of the present disclosure, each of the strip-shaped coupling holes is configured to have a sliding origination and a sliding destination, and the positioning pin is configured to linearly reciprocate between the sliding origination and the sliding destination; as the positioning pin moves, the intersection is formed at different positions co-defined by the strip-shaped guiding portion and one of the two strip-shaped coupling holes, and the positioning pin is configured to slidably couple with different positions of the strip-shaped guiding portion, such that each of the flow-guiding sheets pivots relative to the frame to cause each of the openings to be in the open-up state or the closed state.

In the filtering mask of embodiments of the present disclosure, the first filter pack is provided with the filtration cartridge, the first filter pack is provided with the filtration cartridge, the two ventilation portions, the conduit, and the mask tube, wherein the filtration cartridge is disposed between the two ventilation portions, and the filtration cartridge is coupled in fluid communication with the conduit; the second filter pack is provided with the filtration medium and the wrapper, wherein the filtration medium is disposed within the wrapper that is arranged around the conduit and the mask tube, and the second filter pack and the first filter pack are configured to be movably combined; and the supporting member is detachably disposed between the first filter pack and the second filter pack, such that the filtering mask has functions of being equipped within an air purification device and worn on a human face, in addition to providing ambient air purification function, an air filtering mask for a disaster can also be stocked for emergency needs.

### BRIEF DESCRIPTION OF THE DRAWINGS

FIG. 1 is a schematic diagram showing a coupled perspective view illustrating a filtering mask according to an embodiment of the present disclosure.

FIG. 2 is a schematic diagram showing a partially exploded perspective view illustrating a filtering mask according to an embodiment of the present disclosure.

FIG. 3 is a schematic diagram illustrating the first configuration mode of the filtering mask according to an embodiment of the present disclosure.

FIG. 4 is a schematic diagram illustrating the second configuration mode of the filtering mask according to an embodiment of the present disclosure.

FIG. 5 is a schematic diagram showing an exploded view illustrating a filtering mask according to an embodiment of the present disclosure.

FIG. 6 is a schematic diagram illustrating a first flow-guiding sheet in a hiding state according to an embodiment of the present disclosure.

FIG. 7 is a schematic diagram illustrating the first flow-guiding sheet in an open-up state according to an embodiment of the present disclosure.

FIG. 8 is a schematic diagram illustrating a second flow-guiding sheet in a hiding state according to an embodiment of the present disclosure.

FIG. 9 is a schematic diagram illustrating the second flow-guiding sheet in an open-up state according to an embodiment of the present disclosure.

FIG. 10 is a schematic diagram illustrating a filtering mask equipped with an air purification device according to an embodiment of the present disclosure.

FIG. 11 is a schematic diagram illustrating a filtering mask worn on a human face according to an embodiment of the present disclosure.

### THE DETAILED DESCRIPTION OF PREFERRED EMBODIMENTS

To make the above and other objects, features, and advantages of the present disclosure more comprehensible, preferred embodiments will be exemplified below in detail, together with attached drawings. Furthermore, directional terms mentioned in the present disclosure, such as upper, lower, top, bottom, front, rear, left, right, inner, outer, side, surrounding, central, horizontal, lateral, vertical, longitudinal, axial, radial, uppermost layer or lowermost layer, which only refer to the direction of drawings. Therefore, the directional terms configured above are for illustration and understanding of the present disclosure and are not intended to limit the present disclosure. It should be understood that the terms first and second are used for descriptive purposes only and cannot be interpreted as indicating or implying relative importance or implicitly indicating the quantity of indicated technical features. Thus, a feature defined as first or second may explicitly or implicitly include one or more of said features. In the description of the present disclosure, the meaning of at least one includes one and more than one unless otherwise explicitly defined.

Herein, it should be understood that “fluid communication” means that two components have a structure that can be coupled to each other and allow fluid (such as air or liquid) to communicate with each other, e.g., openings of two hollow members can be coupled or approach each other, such that fluid can be guided to flow inside the two hollow members; “fluid-closed” means that a member is constructed to make fluid such as air or liquid be enclosed and presented in a state of being unable to circulation.

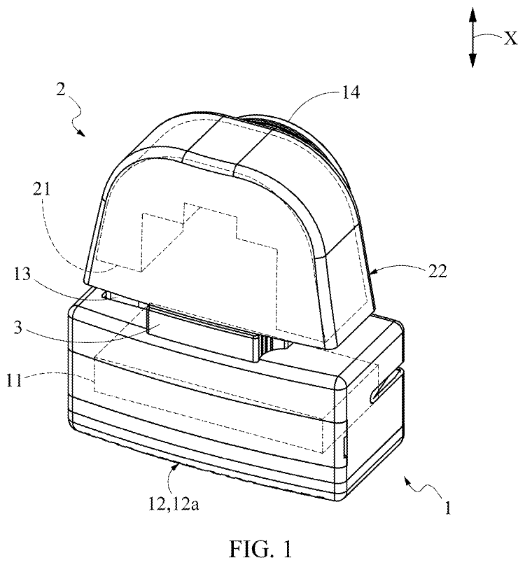

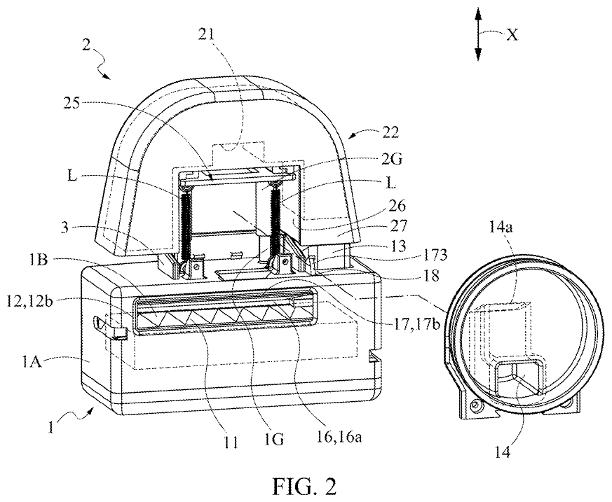

Please refer to FIGS. 1 and 2, one aspect of the present disclosure provides a filtering mask, which is an air filtering device suitable for being equipped with an air purification device (e.g., a single filter pack can be used for fixed-location ventilation) and worn on a human face (e.g., dual filter assemblies can be used to be worn for filtering air). The following examples illustrate implementations of the filtering mask of the embodiment of the present disclosure mentioned above, but they are not limited to the description here.

For example, as shown in FIGS. 1 and 2, the filtering mask includes a first filter pack 1 and a second filter pack 2. For example, the first filter pack 1 is provided with a filtration cartridge 11, two ventilation portions 12, a conduit 13, and a mask tube 14. The filtration cartridge 11 includes, but is not limited to, a high-efficiency particulate air (HEPA) filtration cartridge. The filtration cartridge 11 is disposed between the two ventilation portions 12, and the filtration cartridge 11 is coupled in fluid communication with the conduit 13. The second filter pack 2 is provided with a filtration medium 21 and a wrapper 22. The filtration medium 21 includes, but is not limited to, filtration mediums for preventing or filtering poisonous gas. The filtration medium 21 is disposed within the wrapper 22, and the wrapper 22 is arranged around the conduit 13 and the mask tube 14. The second filter pack 2 and the first filter pack 1 are configured to be movably combined. For example, the first filter pack 1 and the second filter pack 2 can be movably combined in a reciprocating manner along a linear trajectory of an axis X, e.g., adopting configurations such as sliding rails, sleeves, or guiding posts. It is not limited to the description here; in other implementations, the first filter pack 1 and the second filter pack 2 can also be movably combined in a reciprocating manner along a nonlinear trajectory, such that the first filter pack 1 and the second filter pack 2 can be presented in different configuration modes.

As shown in FIGS. 1 and 2, the filtering mask also includes at least one supporting member 3. The supporting member 3 is detachably disposed between the first filter pack 1 and the second filter pack 2. For example, in a configuration mode, the first filter pack 1 and the second filter pack 2 can be held apart from each other by sandwiching the supporting member 3, i.e., the supporting member 3 is sandwiched between the first filter pack 1 and the second filter pack 2. In addition, in another configuration mode, the supporting member 3 can be removed from a space between the first filter pack 1 and the second filter pack 2, such that the supporting member 3 is away from the first filter pack 1 and the second filter pack 2.

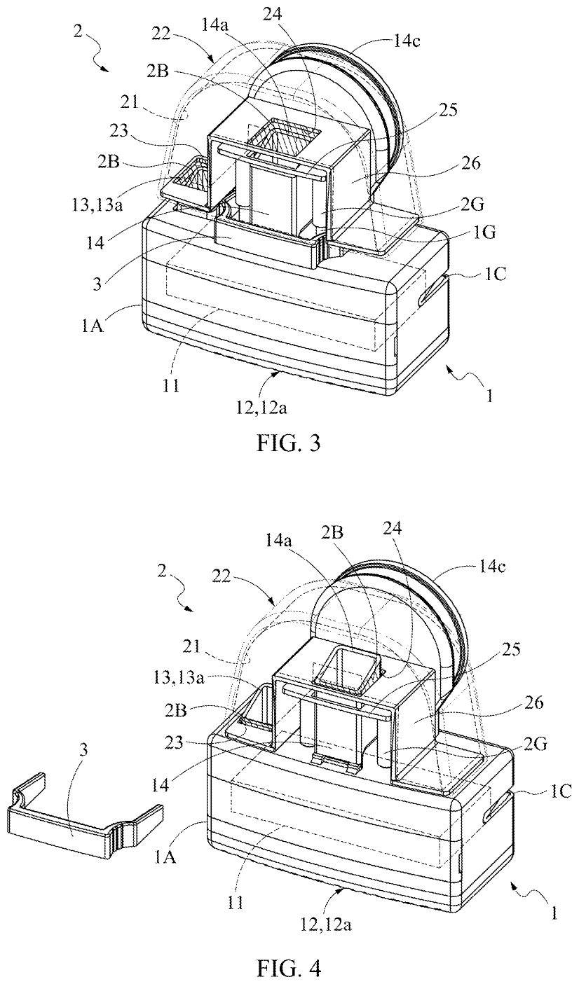

For example, as shown in FIGS. 1, 2, and 3, in a first configuration mode, the supporting member 3 is disposed between the first filter pack 1 and the second filter pack 2, and the first filter pack 1 and the second filter pack are held apart from each other by sandwiching the supporting member 3. For example, the filtration medium 21 is disposed within the wrapper 22 in a fluid-closed manner, and the wrapper 22 is arranged around the conduit 13 and the mask tube 14. For example, one end 13a of the conduit 13 and one end 14a of the mask tube 14 are configured to be toward different parts of the wrapper 22, respectively.

For example, as shown in FIGS. 1, 2, and 4, in a second configuration mode, the supporting member 3 is removed from between the first filter pack 1 and the second filter pack 2, such that the supporting member 3 is away from the first filter pack 1 and the second filter pack 2. For example, the wrapper 22 is affected to move toward the conduit 13 and the mask tube 14 by gravity, such that the conduit 13 and the mask tube 14 break the wrapper 22. A breaking manner can be physical breaks that mean to include making at least one damaged portion such as a hole or a crack in a process such as a breakthrough, extrusion, or push-pull. In one example, one end 13a of the conduit 13 and one end 14a of the mask tube 14 may be toward the same direction, so as to easily apply force to break the wrapper 22, such that the filtration medium 21 is disposed within the wrapper 22 in a non-fluid-closed manner. For example, along a specific direction, the wrapper 22 and the conduit 13 are driven to approach each other, and the wrapper 22 and the mask tube 14 approach each other. For example, the supporting member 3 is removed from a space between the first filter pack 1 and the second filter pack 2 to make the wrapper 22 move down to the conduit 13 and the mask tube 14, wherein a distance for moving the wrapper 22 is enough to make the conduit 13 and the mask tube 14 apply destructive force to the wrapper 22, such that the conduit 13 and the mask tube 14 can cause breakthroughs of the wrapper 22, and the filtration medium 21 is not damaged. It is not limited to the description here; in other implementations, the conduit 13 and the mask tube 14 can move toward the wrapper 22, such that the conduit 13 and the mask tube 14 break through the wrapper 22, and the filtration medium 21 is coupled in fluid communication between the conduit 13 and the mask tube 14.

The example configuration of the filtering mask is illustrated below to facilitate readers' better understanding of the specific content of the present disclosure. Still, it is not intended to limit the present disclosure.

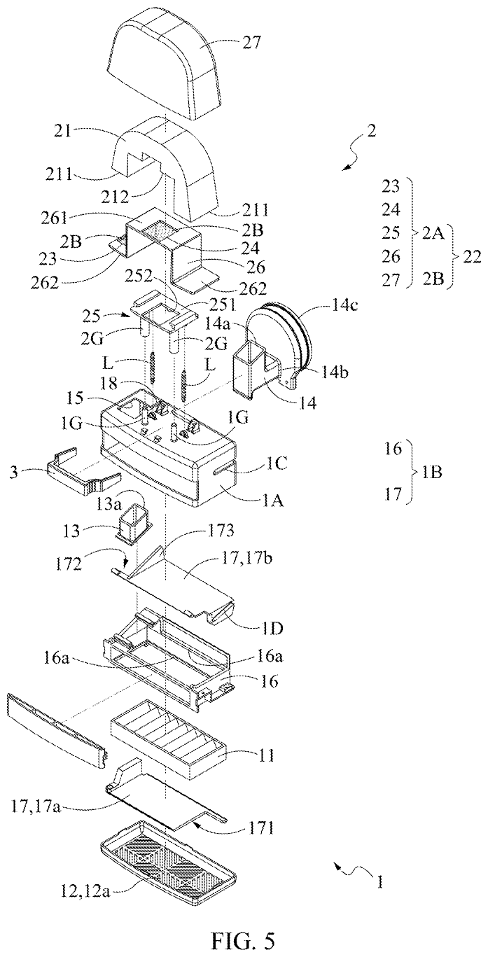

For example, as shown in FIGS. 1, 2, and 5, the first filter pack 1 includes a housing 1A, wherein the filtration cartridge 11 is disposed in the housing 1A, and the two ventilation portions 12 are defined on the housing in different directions. The housing 1A is defined with a first circulation port 15. The first circulation port 15 is coupled with the conduit 13 in fluid communication.

Optionally, as shown in FIGS. 1, 2, and 5, in one embodiment, the two ventilation portions 12 include an air inlet portion 12a and an air outlet portion 12b. In the first configuration mode, the filtration cartridge 11 is coupled in fluid communication between the air inlet portion 12a and the air outlet portion 12b. For example, by adopting a flow-guiding structure that is properly configured, the air flows in through the air inlet portion 12a, the air passes through the filtration cartridge 11, and then the air flows out through the air outlet portion 12b. In the second configuration mode, the filtration cartridge 11 is coupled in fluid communication between the air inlet portion 12a and the conduit 13. For example, by adopting a flow-guiding structure that is properly configured, the air flows in through the air inlet portion 12a, the air passes through the filtration cartridge 11, and then the air flows out through the conduit 13.

Optionally, as shown in FIGS. 1, 2, and 5, in one embodiment, the first filter pack 1 includes a deformable body 1B. The deformable body 1B is disposed in the housing 1A. The filtration cartridge 11 is disposed within the deformable body 1B. The deformable body 1B is configured to be deformable to change a flow rate of the air flowing through the filtration cartridge 11.

Optionally, as shown in FIGS. 1, 2, and 5, in one embodiment, the deformable body 1B includes a frame 16 and two flow-guiding sheets 17. The filtration cartridge 11 is disposed within the frame 16. The frame 16 has two openings 16a. The two openings 16a are adjacent to two sides of the filtration cartridge 11, such as the upper and lower sides of the filtration cartridge 11. The flow-guiding sheets 17 are configured to be pivotably coupled with the frame 16, such that the two openings 16a are in an open-up state or a closed state as the two flow-guiding sheets 17 pivot relative to the frame 16, such as opening or hiding the filtration cartridge 11 for adjusting the flow rate of the air flowing through the filtration cartridge 11.

Optionally, as shown in FIGS. 1 and 2, in one embodiment, the two flow-guiding sheets 17 include a first flow-guiding sheet 17a and a second flow-guiding sheet 17b. The first flow-guiding sheet 17a has a first air delivery portion 171. The second flow-guiding sheet 17b has a second air delivery portion 172. The first air delivery portion 171 and the second air delivery portion 172 are adjacent to two ends of the filtration cartridge 11 (such as the left and right ends of the filtration cartridge 11), respectively. The second air delivery portion 172 and the first circulation port 15 are aligned in the airflow path.

Optionally, as shown in FIGS. 1 and 2, in one embodiment, the second flow-guiding sheet 17b is provided with a baffle 173. The baffle 173 is arranged around the second air delivery portion 172. When the two openings 16a are operated in the closed state, the baffle 173 is used to block the air from the air inlet portion 12a from flowing to the air outlet portion 12b and to assist in guiding the air to the conduit 13. The housing 1A is also defined with a slit 18. The slit 18 and the baffle 173 are aligned with each other. When the two openings 16a are operated in the open-up state, the baffle 173 can run through the housing 1A. In a first configuration mode, the first flow-guiding sheet 17a and the second flow-guiding sheet 17b cause the two openings 16a to be operated in the open-up state, in which the baffle 173 passes through the slit 18, and the filtration cartridge 11 is coupled in fluid communication between the two ventilation portions 12, such that the air can longitudinally flow from one of the ventilation portions 12 to the other of the ventilation portions 12 through the filtration cartridge 11. In the second configuration mode, the first flow-guiding sheet 17a and the second flow-guiding sheet 17b cause the two openings 16a to be operated in the closed state, in which the filtration cartridge 11 is coupled in fluid communication between the first air delivery portion 171 and the second air delivery portion 172, such that the air can laterally flow from the first air delivery portion 171 to the second air delivery portion 172 through the filtration cartridge 11. Thus, the air is concentrated and guided to the filtration medium 21 and the mask tube 14 through the conduit 13.

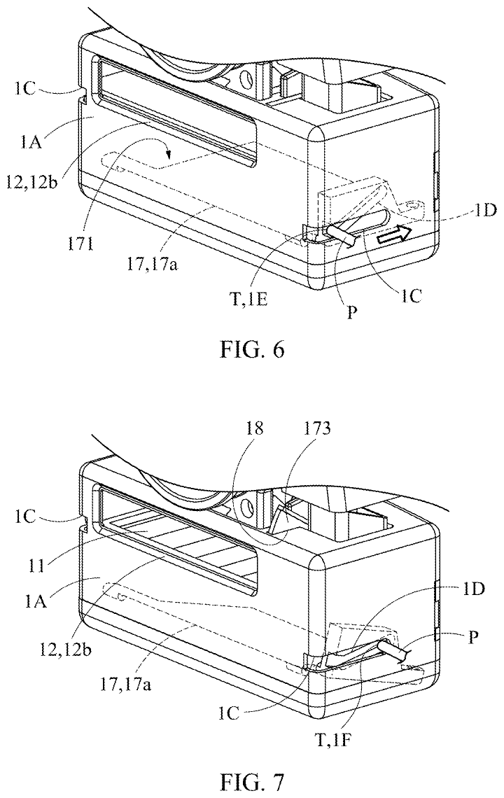

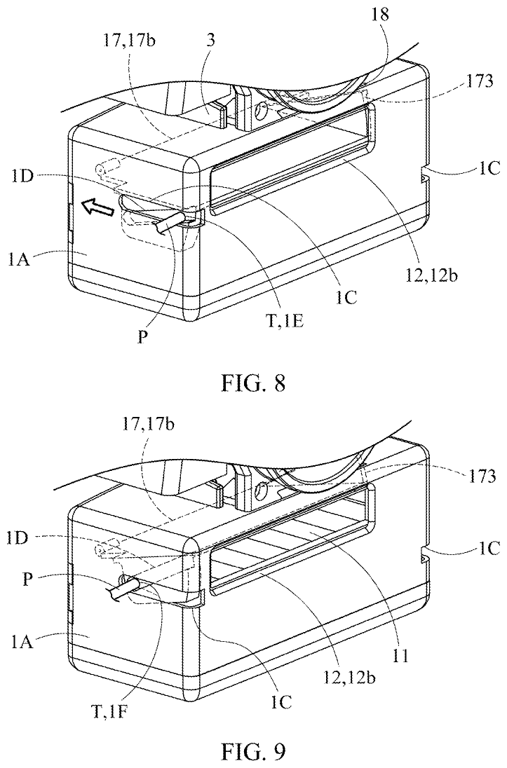

Optionally, as shown in FIGS. 6 to 9, in one embodiment, the housing 1A may be formed with two strip-shaped coupling holes 1C, the first flow-guiding sheet 17a may be formed with a strip-shaped guiding portion 1D (as shown in FIGS. 6 and 7), and the second flow-guiding sheet 17b can be formed with a strip-shaped guiding portion 1D (as shown in FIGS. 8 and 9). For example, a single strip-shaped coupling hole 1C and a single strip-shaped guiding portion 1D are aligned with each other in a lateral direction to form a sliding configuration. Features (such as shape and position) of the strip-shaped guiding portion 1D and strip-shaped coupling holes 1C between different sliding configurations can be fine-tuned according to actual needs. Each of the strip-shaped coupling holes 1C is configured to allow a positioning pin P to pass through slidably, and the positioning pin P is slidably combined to the strip-shaped guiding portion 1D, such that an intersection T is formed between the strip-shaped guiding portion 1D and one of the strip coupling holes 1C.

Optionally, as shown in FIGS. 6 to 9, in one embodiment, each strip-shaped coupling hole 1C has a sliding origination 1E and a sliding destination 1F, and the positioning pin P is configured to linearly reciprocate between the sliding origination 1E and the sliding destination 1F. As the positioning pin P moves, the intersection T is formed at different positions co-defined by the strip-shaped guiding portion 1D and one of the two strip-shaped coupling holes 1C, and the positioning pin P is configured to slidably couple with different positions of the strip-shaped guiding portion 1D, such that each of the flow-guiding sheets 17 pivots relative to the frame 16 to cause each of the openings 16a to be in the open-up state or the closed state.

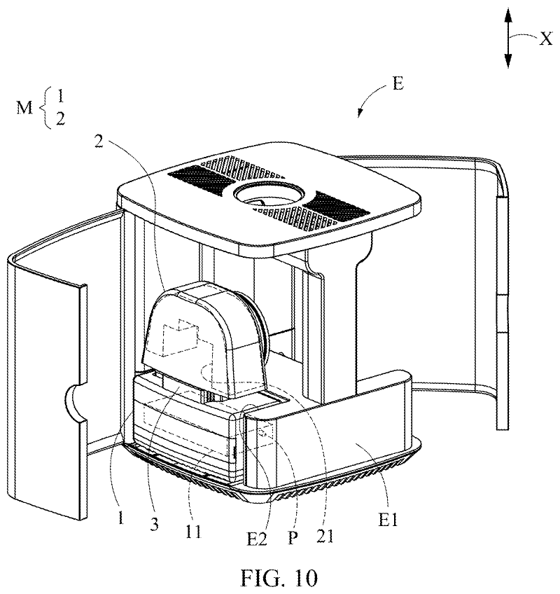

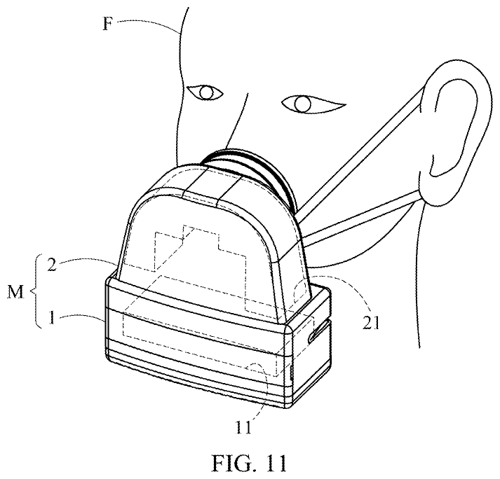

Optionally, as shown in FIG. 10, in one embodiment, the positioning pin P can be disposed within an accommodating recess, such as a recess inside an air purification device E. The accommodating recess is configured to be detachably disposed in the housing 1A. Please refer to FIGS. 6 to 9, when the housing 1A is assembled in the air purification device E, such as pushing the housing 1A into the air purification device E, such that the positioning pin P moves from the sliding origination 1E to the sliding destination 1F. Thus, the positioning pin P can assist the flow-guiding sheet 17 to automatically pivot from the closed state to the open-up state, such that the filtration cartridge 11 can be used to provide an air filtering function. When the housing 1A is disassembled from the air purification device E, e.g., the housing 1A is pulled out of the air purification device E, such that the positioning pin P moves from the sliding destination 1F to the sliding origination 1E. Thus, the positioning pin P can assist the flow-guiding sheet 17 to automatically pivot from the open-up state to the closed state, in which the dust near the filtration cartridge 11 can be prevented from being scattered. It is not limited to the description here; in other implementations, the accommodating recess can also be a recess inside a packaging box, and the positioning pin P can assist the flow-guiding sheet 17 to be maintained in the closed state, so as to facilitate transportation or storage operations.

Optionally, as shown in FIGS. 1, 2, and 5, in one embodiment, in the second filter pack 2, the wrapper 22 includes a packaging assembly 2A and two sealing membranes 2B, wherein the filtration medium 21 is disposed in the packaging assembly 2A. The packaging assembly 2A is defined with a second circulation port 23 and a third circulation port 24. The second circulation port 23 and the conduit 13 are aligned in an airflow path, and the third circulation port 24 and the mask tube 14 are aligned in the airflow path. In one example, all of the filtration cartridge 11, the first circulation port 15, the conduit 13, the second circulation port 23, the filtration medium 21, the third circulation port 24, and the mask tube 14 are located in the airflow path. The two sealing membranes 2B can be two independent membranes or two portions of a single membrane. The two sealing membranes 2B are mounted on the second circulation port 23 and the third circulation port 24, respectively. For example, metal or plastic membranes can be disposed at the second circulation port 23 and the third circulation port 24 to assist in the realization that the filtration medium 21 is disposed within the wrapper 22 in a fluid-closed manner, but it is not limited to the description here. In other implementations, the two sealing membranes 2B may also be a local structural-fragile-thin portion formed by the material of the wrapper 22.

Optionally, as shown in FIGS. 1, 2, and 5, in one embodiment, the filtration medium 21 is shaped as an arc-shaped filtration medium, in which a connection portion 212 is provided between two ends 211 of the arc-shaped filtration medium. The conduit 13 has one end 13a toward one of the two ends 211 of the arc-shaped filtration medium, and the mask tube 14 has one end 14a toward the connection portion 212 of the arc-shaped filtration medium.

Optionally, as shown in FIGS. 1, 2, and 4, in one embodiment, in the second configuration mode, the wrapper 22 gradually approaches the conduit 13 and the mask tube 14 until the conduit 13 makes a breakthrough of the sealing membrane 2B provided at the second circulation port 23, and the mask tube 14 makes a breakthrough of the sealing membrane 2B provided at the third circulation port 24, such that the filtration medium 21 is coupled in fluid communication between the conduit 13 and the mask tube 14.

Optionally, as shown in FIGS. 1, 2, and 4, in one embodiment, one of two ends of the conduit 13 is configured to form a first tip 13a toward the second circulation port 23 to assist in puncturing break the sealing membrane 2B provided at the second circulation port 23; one of two ends of the mask tube 14 is configured to form a second tip 14a toward the third circulation port 24 to assist in breaking the sealing membrane 2B provided at the third circulation port 24 in a puncturing manner.

Optionally, as shown in FIGS. 1, 2, and 5, in one embodiment, the other end 14b of the mask tube 14 is provided with a breathing aid 14c. For example, an appropriate connection structure, such as a tube with a straight structure, a curved structure, or a turning structure, can be formed between the two ends 14a and 14b of the mask tube 14. In addition, the breathing aid 14c can be configured as an air delivery device for wearing on the respiratory facial features (such as the mouth or nose) of the human face, such as an emergency escape mask, a nasal pillow mask tube, or a nasal mask tube. It is not limited to the description here; in other implementations, the breathing aid 14c can also be an air supply sleeve without a mask or a combination of an air supply sleeve and a mask.

Optionally, as shown in FIGS. 1, 2, and 5, in one embodiment, the housing 1A is provided with a plurality of first move-guiding elements 1G, and the packaging assembly 2A includes a move-guiding base 25, a seat 26, and a cover 27, wherein the move-guiding base has a plurality of second move-guiding elements 2G, and the plurality of first move-guiding elements 1G and the plurality of second move-guiding elements 2G are slidably combined. For example, the plurality of first move-guiding elements 1G and the plurality of second move-guiding elements 2G can be adopted in the form of a movable joint structure such as a sleeve or a guiding post, but it is not limited to the description here. The plurality of first move-guiding elements 1G and the plurality of second move-guiding elements 2G can also be adopted in the form of different parts of a slide rail, such that the packaging assembly 2A and the housing 1A can reciprocate along an axis X. The seat 26 is mounted on the move-guiding base 25 in a straddling manner and carries the filtration medium 21. The cover 27 is configured to cover the filtration medium 21 and coupled with the seat 26.

Optionally, as shown in FIGS. 1, 2, and 5, in one embodiment, the move-guiding base 25 also has a substrate 251, wherein the substrate 251 is connected to the plurality of second move-guiding elements 2G. The substrate 251 is also defined with a through hole 252, wherein the through hole 252 and the third circulation port 24 are aligned in the airflow path. When the plurality of first move-guiding elements 1G and the plurality of second move-guiding elements 2G linearly move relative to each other, such as making the third circulation port 24 and one end 14a of the mask tube 14 approach each other gradually, one end 14a of the mask tube 14 can pass through the through hole 252 and the third circulation port 24 for breaking the sealing membrane 2B provided at the third circulation port 24.

Optionally, as shown in FIGS. 1, 2, and 5, in one embodiment, at least one elastic element L, such as a spring or an elastic band, can also be disposed between the housing 1A and the substrate 251. The at least one elastic element L is arranged around the plurality of first move-guiding elements 1G and the plurality of second move-guiding elements 2G to prevent a gap between the first filter pack 1 and the second filter pack 2 from being too long.

Optionally, as shown in FIGS. 1, 2, and 5, in one embodiment, the seat 26 has a top 261 and two shoulders 262, wherein the second circulation port 23 is defined on one of the two shoulders 262, and the third circulation port 24 is defined on the top 261. In this way, the seat is convenient for carrying the filtration medium and providing the two sealing membranes.

The filtering mask of the embodiments mentioned above of the present disclosure can be applied to many usage scenarios. The examples are provided as follows but are not limited to the description here.

For example, as shown in FIGS. 2, 3, and 10, in one example, the use of a filtering mask M includes, but is not limited to, disposing the filtering mask M in an air purification device E. For example, the filtration cartridge 11 of the first filter pack 1 of the filtering mask M is provided in a first air-filtering path inside the air purification device E. For example, the air purification device E can be provided in an indoor environment, and the air purification device E can be equipped with the filtering mask M. For example, a housing 1A of the first filter pack 1 is detachably disposed within an accommodating recess E2 of a casing E1 of the air purification device E. A supporting member 3 can be provided between the first filter pack 1 and a second filter pack 2. In the first configuration mode, the filtration medium 21 of the second filter pack 2 is disposed within the wrapper 22 in a fluid-closed manner, and the wrapper 22 is arranged around the conduit 13 and the mask tube 14 of the first filter pack 1. In the first filter pack 1, the filtration cartridge 11 is coupled in fluid communication between two ventilation portions 12. For example, unfiltered air enters through the air inlet portion 12a, the air passes through the filtration cartridge 11 longitudinally to filter dust, and then the filtered air is blown out from the air outlet portion 12b. Thus, because the filtration cartridge is located in the first air-filtering path within the air purification device, it can provide an ambient air purification function.

For example, as shown in FIGS. 4, 5, and 11, in one example, the use of a filtering mask M includes, but is not limited to, wearing the filtering mask M on a human face F. For example, the filtration cartridge 11 of the first filter pack 1 and the filtration medium 21 of the second filter pack 2 of the filter mask M are provided in a second air-filtering path outside the human face F. For example, a user can take out the filtering mask M from an air purification device E in a smoky environment. The supporting member 3 can be removed between the first filter pack 1 and the second filter pack 2, such that the second filter pack 2 is driven to naturally fall by gravity, forcing the conduit 13 and the mask tube 14 to physically break the wrapper 22, as in the second configuration mode. For example, the conduit 13 and the mask tube 14 make breakthroughs of the two sealing membranes 2B, causing the filtration medium 21 to be coupled in fluid communication between the conduit 13 and the mask tube 14. In the first filter pack 1, the filtration cartridge 11 is coupled in fluid communication between the first air delivery portion 171 and the second air delivery portion 172. For example, unfiltered air enters through an air inlet portion 12a, the air can pass through the filtration cartridge 11 laterally from the first air delivery portion 171 to filter dust, the filtered air then enters the conduit 13 from the second air delivery portion 172, and the filtered air passes through the filtration medium 21 from the conduit 13 to filter or absorb harmful substances, and then the filtered air is sent out by the mask tube 14. In this way, the filtering mask can be worn on the human face; for example, the human face wears the breathing aid. Because the filtration cartridge and the filtration medium are located in the second air-filtering path outside the human face, the air passing through the filtration cartridge and the filtration medium is filtered in two stages. The user can use the filtering mask to inhale the filtered air, which can be applied to the fire and smoke prevention process to avoid human casualties caused by the inhalation of toxic gases.

According to the air purification device equipped with the filtering mask according to the above embodiment of the present disclosure, the filtration cartridge, the two ventilation portions, the conduit, and the mask tube are provided in the first filter pack, the filtration cartridge is disposed between the two ventilation portions, and the filtration cartridge is coupled in fluid communication with the conduit; the second filter pack is provided with the filtration medium and the wrapper, the filtration medium is disposed within the wrapper, and the second filter pack and the first filter pack are configured to be movably combined; and the supporting member is detachably disposed between the first filter pack and the second filter pack, such that the filtering mask has functions of being equipped within an air purification device and worn on a human face, in addition to providing ambient air purification function, an air filtering mask for a disaster can also be stocked for emergency needs.

Although the present disclosure has been disclosed in preferred embodiments, which are not intended to limit the disclosure, those skilled in the art can make various changes and modifications without departing from the spirit and scope of the disclosure. Therefore, the scope of protection of the present disclosure is defined as definitions of the scope of the claims.

What is claimed is:

1. A filtering mask, comprising:
a first filter pack provided with a filtration cartridge, two ventilation portions, a conduit, and a mask tube, wherein the filtration cartridge is disposed between the two ventilation portions, and the filtration cartridge is coupled in fluid communication with the conduit;
a second filter pack provided with a filtration medium and a wrapper, wherein the filtration medium is disposed within the wrapper that is arranged around the conduit and the mask tube, and the second filter pack and the first filter pack are configured to be movably combined; and
a supporting member detachably disposed between the first filter pack and the second filter pack.

2. The filtering mask as claimed in claim 1, wherein in a first configuration mode, the supporting member is disposed between the first filter pack and the second filter pack, and the filtration medium is disposed within the wrapper in a fluid-closed manner; in a second configuration mode, the supporting member is away from the first filter pack and the second filter pack, and the conduit and the mask tube break the wrapper to make the filtration medium be coupled in fluid communication between the conduit and the mask tube.

3. The filtering mask as claimed in claim 2, wherein the two ventilation portions comprise an air inlet portion and an air outlet portion, and in the first configuration mode, the filtration cartridge is coupled in fluid communication between the air inlet portion and the air outlet portion; in the second configuration mode, the filtration cartridge is coupled in fluid communication between the air inlet portion and the conduit.

4. The filtering mask as claimed in claim 1, wherein the filtration medium is shaped as an arc-shaped filtration medium, in which a connection portion is provided between two ends of the arc-shaped filtration medium, wherein one end of the conduit is configured to be toward one of the two ends of the arc-shaped filtration medium, and one end of the mask tube is configured to be toward the connection portion of the arc-shaped filtration medium.

5. The filtering mask as claimed in claim 1, wherein the first filter pack comprises a housing, the filtration cartridge is disposed in the housing, the two ventilation portions are defined on the housing in different directions, and the housing is defined with a first circulation port that is coupled with the conduit in fluid communication; the wrapper comprises a packaging assembly and two sealing membranes, the filtration medium is disposed within the packaging assembly, and the packaging assembly is defined with a second circulation port and a third circulation port, wherein the second circulation port and the conduit are aligned in an airflow path, the third circulation port and the mask tube are aligned in the airflow path, and the two sealing membranes are mounted on the second circulation port and the third circulation port.

6. The filtering mask as claimed in claim 5, wherein the housing is provided with a plurality of first move-guiding elements, and the packaging assembly comprises a move-guiding base, a seat, and a cover, wherein the move-guiding base has a plurality of second move-guiding elements that are slidably combined with the plurality of first move-guiding elements, the seat is mounted on the move-guiding base in a straddling manner and carries the filtration medium, and the cover covers the filtration medium and is coupled with the seat.

7. The filtering mask as claimed in claim 6, wherein the move-guiding base has a substrate that is connected to the plurality of second move-guiding elements and defined with a through hole, wherein the through hole and the third circulation port are aligned in the airflow path, and during the plurality of first move-guiding elements and the plurality of second move-guiding elements linearly move relative to each other, one end of the mask tube passes through the through hole and the third circulation port.

8. The filtering mask as claimed in claim 6, wherein the seat has a top and two shoulders, the second circulation port is defined on one of the two shoulders, and the third circulation port is defined on the top.

9. The filtering mask as claimed in claim 7, wherein at least one elastic element is disposed between the housing and the substrate, and the at least one elastic member is arranged around the plurality of first move-guiding elements and the plurality of second move-guiding elements.

10. The filtering mask as claimed in claim 5, wherein one of two ends of the conduit is configured to form a first tip toward the second circulation port; one of two ends of the mask tube is configured to form a second tip toward the third circulation port.

11. The filtering mask as claimed in claim 10, wherein the other of the two ends of the mask tube is provided with a breathing aid.

12. The filtering mask as claimed in claim 5, wherein the first filter pack comprises a deformable body, the deformable body is disposed in the housing, the filtration cartridge is disposed within the deformable body, and the deformable body is configured to be deformable to change a rate of air flowing through the filtration cartridge.

13. The filtering mask as claimed in claim 12, wherein the deformable body comprises a frame and two flow-guiding sheets, and the filtration cartridge is disposed within the frame that has two openings adjacent to two sides of the filtration cartridge, wherein the two flow-guiding sheets are configured to be pivotally connected to the frame, and the two openings are in an open-up state or a closed state as the two flow-guiding sheets pivot relative to the frame.

14. The filtering mask as claimed in claim 13, wherein the two flow-guiding sheets comprise a first flow-guiding sheet and a second flow-guiding sheet, wherein the first flow-guiding sheet is defined with a first air delivery portion, the second flow-guiding sheet is defined with a second air delivery portion, the first air delivery portion and the second air delivery portion are adjacent to the two ends of the filtration cartridge, and the second air delivery portion and the first circulation port are aligned in the airflow path.

15. The filtering mask as claimed in claim 14, wherein the housing is defined with a slit, and the second flow-guiding sheet is provided with a baffle that is arranged around the second air delivery portion; in a first configuration mode, the first flow-guiding sheet and the second flow-guiding sheet cause the two openings to be in the open-up state, the baffle passes through the slit, and the filtration cartridge is coupled in fluid communication between the two ventilation portions; in a second configuration mode, the first flow-guiding sheet and the second flow-guiding sheet cause the two openings to be in the closed state, and the filtration cartridge is coupled in fluid communication between the first air delivery portion and the second air delivery portion.

16. The filtering mask as claimed in claim 13, wherein the housing is defined with two strip-shaped coupling holes, and the two flow-guiding sheets are respectively provided with a strip-shaped guiding portion, each of the two strip-shaped coupling holes is configured to allow a positioning pin to pass through slidably, and the positioning pin is slidably combined with the strip-shaped guiding portion, such that an intersection is formed between the strip-shaped guiding portion and one of the two strip-shaped coupling holes.

17. The filtering mask as claimed in claim 16, wherein each of the strip-shaped coupling holes is configured to have a sliding origination and a sliding destination, and the positioning pin is configured to linearly reciprocate between the sliding origination and the sliding destination; as the positioning pin moves, the intersection is formed at different positions co-defined by the strip-shaped guiding portion and one of the two strip-shaped coupling holes, and the positioning pin is configured to slidably couple with different positions of the strip-shaped guiding portion, such that each of the flow-guiding sheets pivots relative to the frame to cause each of the openings to be in the open-up state or the closed state.
